(19) United States
(12) Patent Application Publication
(10) Pub. No.: US 2019/0232007 A1
(43) Pub. Date: Aug. 1, 2019

(54) ENDOTRACHEAL TUBE STYLET

(71) Applicant: H. LEE MOFFITT CANCER CENTER & RESEARCH INSTITUTE, INC., Tampa, FL (US)

(72) Inventor: Tariq Chaudhry, Boston, MA (US)

(21) Appl. No.: 16/330,810

(22) PCT Filed: Sep. 6, 2017
(86) PCT No.: PCT/US17/50169

§ 371 Date: Mar. 6, 2019

Related U.S. Application Data
(60) Provisional application No. 62/383,831, filed on Sep. 6, 2016.

(51) Int. Cl. A61M 16/04

(52) U.S. Cl. CPC A61M 16/0488; A61M 2205/0216; A61M 16/0468

(57) ABSTRACT

Disclosed herein is an endotracheal tube stylet that uses air flow to articulate the stylet and endotracheal tube during intubation. This has the dual advantage of providing oxygen to the patient during intubation while also replacing existing mechanical forms of articulation.

### CROSS REFERENCE TO RELATED APPLICATIONS

This application claims the benefit of priority to U.S. Provisional Application 62/383,831, filed Sep. 6, 2016, which is hereby incorporated by reference herein in its entirety.

### BACKGROUND

Endotracheal tubes are utilized in a wide variety of medical procedures to provide an unobstructed air passage to a patient's trachea. In many emergency situations, it is necessary to intubate a patient as quickly as possible to provide a secure airway to the patient's lungs or permit forced ventilation thereof while preventing introduction of gastric contents. Failure to quickly supply oxygen to the lungs can result in brain damage or death of the patient.

Endotracheal tubes are used orally and nasally to establish an open airway. Intubation is often difficult because of the contours and obstacles encountered in the patient's airway. Perhaps the most difficult step in intubating a patient is maneuvering the tube into the patient's trachea rather than the patient's esophagus.

Endotracheal tubes are generally formed of a soft, pliable plastic materials. Most endotracheal tubes do not have sufficient strength or rigidity to permit intubation without the aid of a stylet or other manipulating device. Making the endotracheal tube out of a stiffer material is not an acceptable alternative because it would cause excessive trauma to the nasal or throat tissue. The accepted solution has been the use of a rigid stylet telescopically received within the endotracheal tube. However, these stylets often do not conform to the anatomical contours of each patient's airway and frequently force the user to abort the intubation, hand-mold the stylet into a different shape and re-attempt the intubation. Multiple intubation attempts in an anesthetized, oxygen starved patient risk airway trauma, injury to the vital organs and in some cases, death.

### SUMMARY

Disclosed herein is an endotracheal tube stylet that uses air flow to articulate the stylet and endotracheal tube during intubation. This has the dual advantage of providing oxygen to the patient during intubation while also replacing existing mechanical forms of articulation.

The disclosed stylet with its soft material and user-adjustable tip conforms to the patient's airway, guides the endotracheal tube into the trachea and overcomes the technical difficulties seen with the traditional, rigid-body stylets. Additionally, oxygen flow through the stylet provides a life-saving advantage by forcing oxygen into the lungs of an otherwise oxygen starved patient.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic of an endotracheal tube stylet according to one implementation.

FIG. 2 is a perspective view of an endotracheal tube stylet telescopically received in an endotracheal tube according to one implementation.

FIG. 3 is a bottom perspective view of a nozzle for an endotracheal tube stylet according to one implementation.

FIG. 4 is a side view of an endotracheal tube stylet according to one implementation.

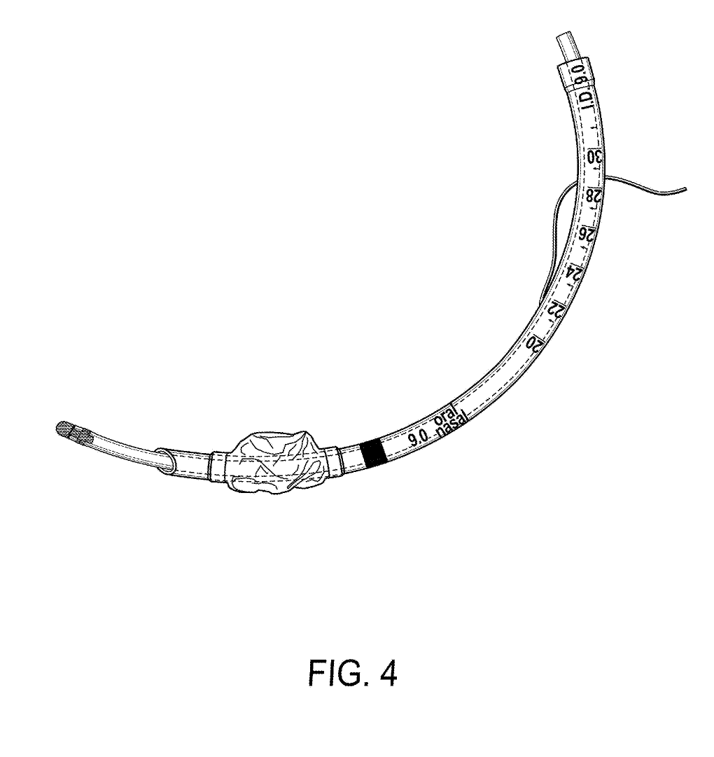

FIG. 5 is a schematic of an endotracheal tube stylet according to one implementation.

FIG. 6 is a schematic of an endotracheal tube stylet according to one implementation.

FIG. 7 is a schematic of an endotracheal tube stylet according to one implementation.

### DETAILED DESCRIPTION

The present invention now will be described more fully hereinafter with reference to specific embodiments of the invention. The invention can be embodied in many different forms and should not be construed as limited to the embodiments set forth herein; rather, these embodiments are provided so that this disclosure will satisfy applicable legal requirements.

As used in the specification, and in the appended claims, the singular forms “a,” “an,” “the,” include plural referents unless the context clearly dictates otherwise.

The term “comprising” and variations thereof as used herein are used synonymously with the term “including” and variations thereof and are open, non-limiting terms.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

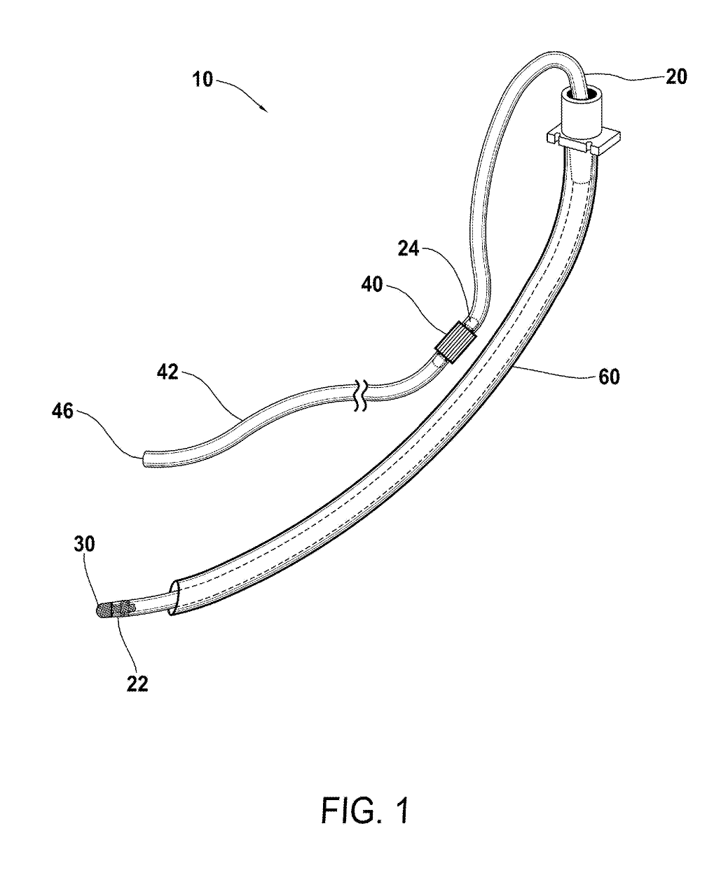

Now referring more particularly to FIG. 1 of the drawings, an embodiment of an endotracheal tube stylet 10 comprises an elongated flexible conduit 20 defining a first air flow path having a proximal end 24 and a distal end 22. The elongated flexible conduit 20 has an inner diameter and an outer diameter. The elongated flexible conduit 20 is sized to be received within an endotracheal tube 60. Therefore, the outer diameter of the elongated flexible conduit 20 is sized to be less than the inner diameter of an endotracheal tube 60. In some embodiments, the outer diameter of the elongated flexible conduit 20 is less than 8 mm, including about 3 to 8 mm. The inner diameter of the elongated flexible conduit 20 can be about 2 to 7 mm.

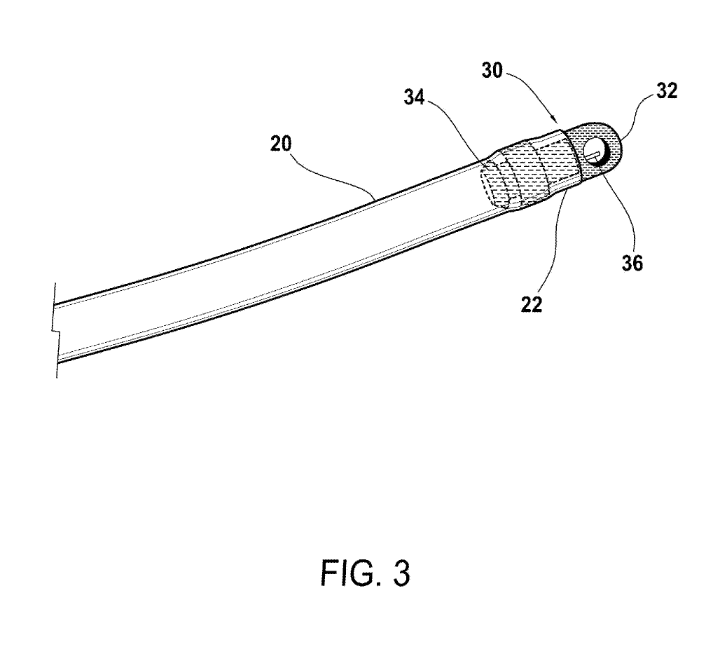

Referring to FIG. 1 and FIG. 3, the distal end 22 of the elongated flexible conduit 20 is affixed to a nozzle 30 having an aperture 36 (FIG. 3) fluidly connected to the first air flow path. The nozzle 30 has a proximal end 34, a distal end 32, a top, and a bottom. The proximal end 34 of the nozzle 30 comprises an inlet port fluidly connecting the air flow path to the aperture 36. The aperture 36 is positioned on the bottom of the nozzle 30 such that the air flow path is redirected downward relative to the orientation of endotracheal tube stylet 10. When air pressure is applied to the first air flow path, the downward flow of air from the nozzle 30 moves the distal end of the elongated flexible conduit 20 in the opposite direction. This articulates the stylet 10, and therefore the endotracheal tube 60 it is disposed within, altering its curvature, which aids in intubation.

The endotracheal tube stylet 10 also contains an air flow valve 40 affixed to the proximal end of the elongated flexible conduit 20 in fluid communication with the first air flow path. The air flow valve 40 is sized and configured for connection to an air flow source, such as pressured oxygen. Therefore, the air flow valve 40 can also be affixed to an air flow source conduit 42 having a distal end 46 configured for connection to an air flow source.

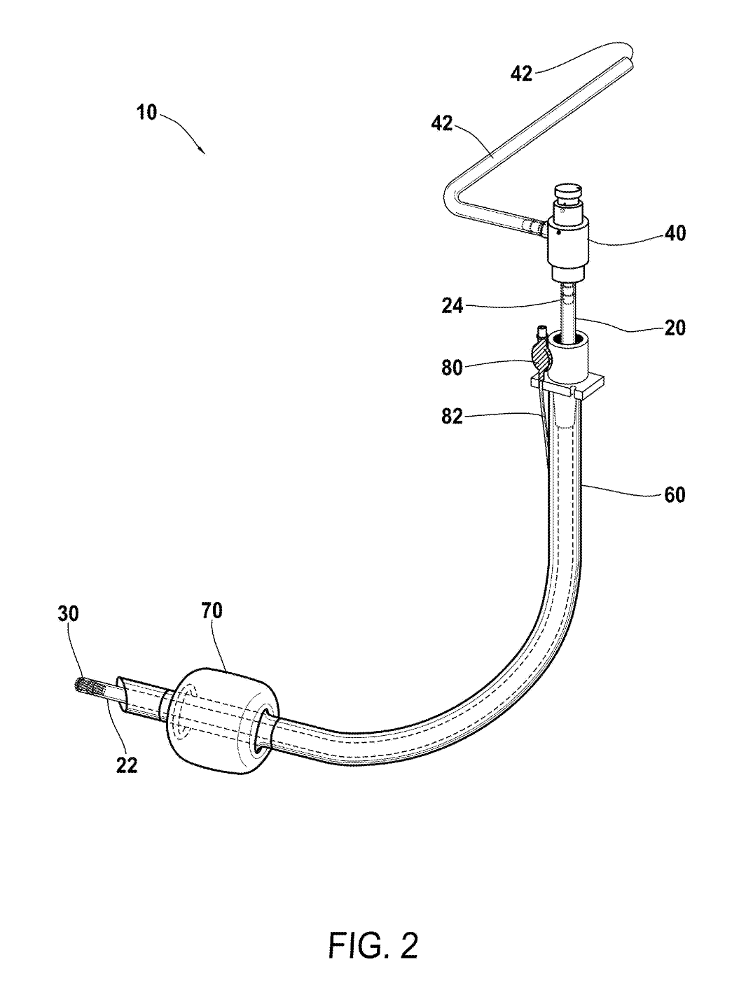

The air flow valve 40 places the air flow source in fluid communication with the air flow path of the elongated flexible conduit 20 when the valve 40 is opened. For example, the air flow valve 40 can include a button, knob, dial, lever, or other such mechanisms for opening the valve 40. For example, as shown in FIG. 2, the valve 40 can be a button configured to be depressed with a thumb or finger while holding the proximal end of an endotracheal tube 60. Alternatively, as depicted in FIG. 1, the valve 40 can be configured to be opened while holding the middle of endotracheal tube 60.

FIG. 2 illustrates an embodiment of an endotracheal tube stylet 10 telescopically received within an endotracheal tube 60. The endotracheal tube 60 tube can have a balloon 70 fluidly connected to an inflation line 82 and optional pilot balloon 80.

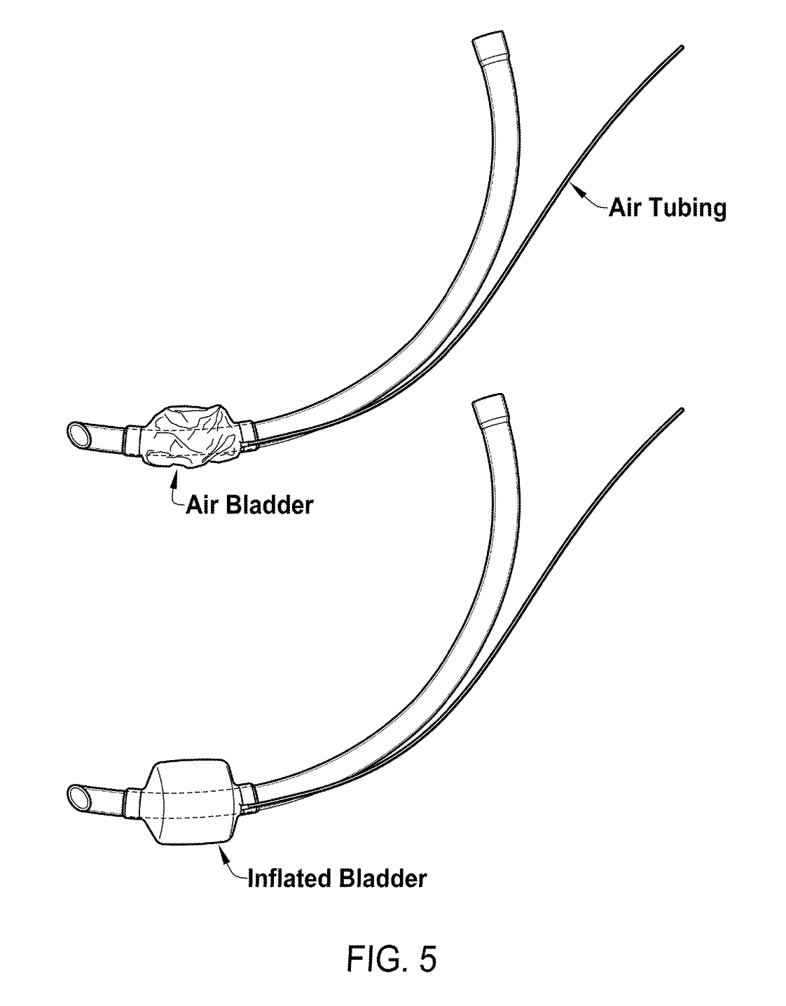

Turning now to FIG. 5, the disclosed stylet 20 can also have a bladder fluidly connected to an inflation device affixed near the distal end 22 of the elongated flexible conduit 20. As with the nozzle 30, the elongated flexible conduit 20 can also have a top and bottom surface, such that the bladder is oriented on the bottom surface. In these embodiments, inflation of the bladder, e.g., with air or water, can divert the distal end 22 of the elongated flexible conduit 20 in an anterior direction.

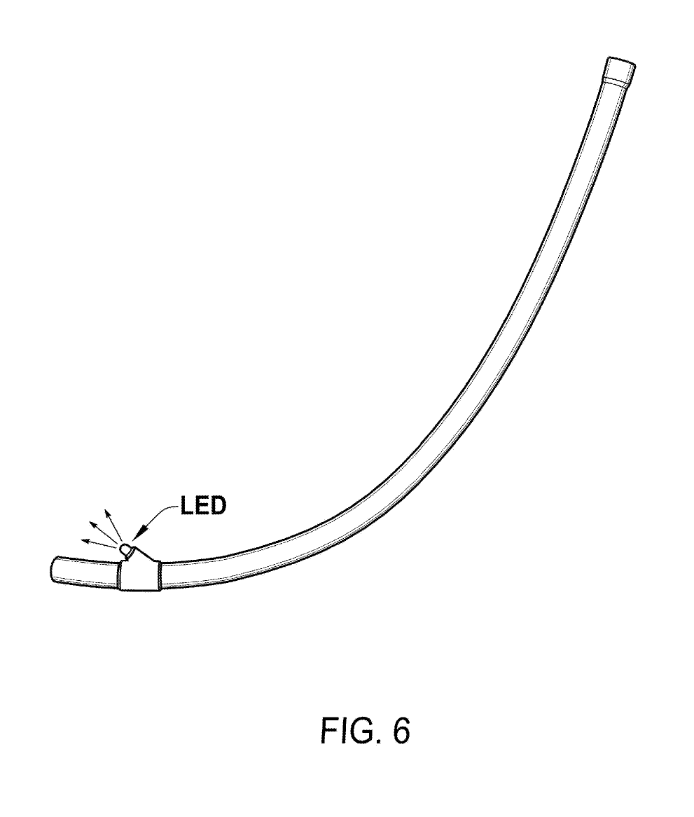

Turning now to FIG. 6, the disclosed stylet 20 can also have an illumination device affixed to the distal end 22 of the elongated flexible conduit 20 to aid in visualization of the trachea.

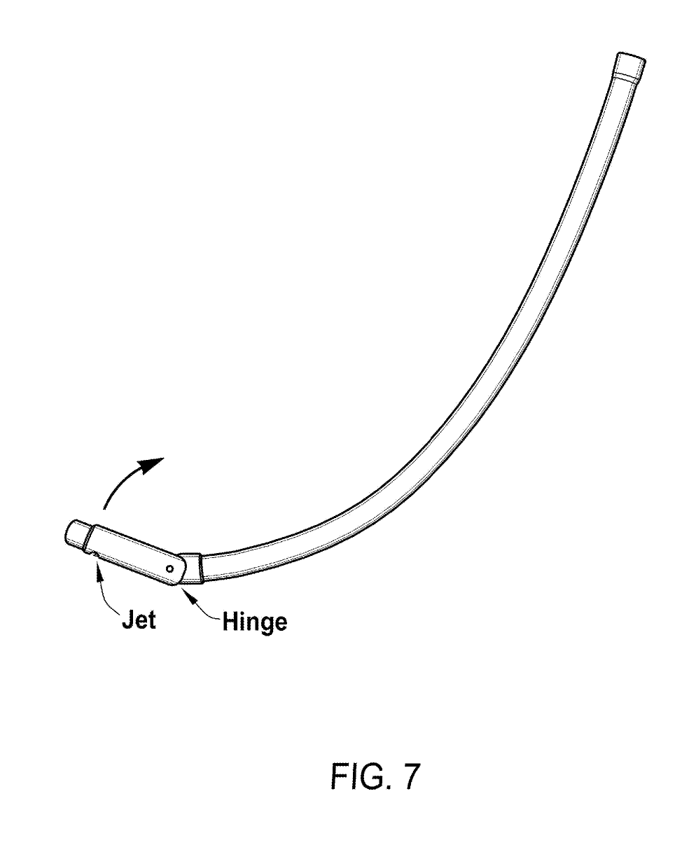

Turning now to FIG. 7, the disclosed stylet 20 can also have one or more mechanical hinges or pivots near the distal end 22 of the elongated flexible conduit 20. For example, a first hinge can be located about 1 to 2 inches from the distal end 22 of the elongated flexible conduit 20. As depicted in FIG. 7, flow through the aperture 36 causes articulation of the distal end 22 of the elongated flexible conduit 20 about the hinge.

In some embodiments, the disclosed elongated flexible conduit 20 defines a second, third, or fourth air flow path fluidly connected to a second, third, or fourth aperture, each of which can be located in the nozzle 30 or in another location on the elongated flexible conduit 20. In these embodiments, air flow through the second, third, or fourth aperture can redirect the distal end 22 of the elongated flexible conduit 20 in a second direction, e.g. lateral or posterior directions. In these embodiments, the air flow valve 40 can also be in fluid communication with the second, third, and fourth air flow path, with separate button, knob, dial, or levers for each air flow path. In other embodiments, the endotracheal tube stylet 10 also contains a second, third, and/or fourth air flow valve affixed to the proximal end of the elongated flexible conduit 20 in fluid communication with the second, third, and/or fourth air flow path.

A system is disclosed that comprises the disclosed endotracheal tube stylet 10 telescopically received within an endotracheal tube 60. The system can be sterile, packaged, and ready for intubation. The system can be removed from the packaging, connected to a source of pressurized air, and then used to intubate a patient. The valve 40 is used to articulate the endotracheal tube 60, e.g., in an anterior direction. Once correctly placed, the balloon 70 can be inflated and the stylet 20 can be removed.

The stylet is configured to use any form of medical gas for patient use. In preferred embodiments, the gas is medical grade oxygen, medical grade air, or some combination thereof.

A number of embodiments of the invention have been described. Nevertheless, it will be understood that various modifications may be made without departing from the spirit and scope of the invention.

Disclosed are materials, systems, devices, compositions, and components that can be used for, can be used in conjunction with, can be used in preparation for, or are products of the disclosed methods, systems and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein.

1. An endotracheal tube stylet, comprising
(a) an elongated flexible conduit defining an air flow path having a proximal end and a distal end,
(b) a nozzle affixed to the distal end of the elongated flexible conduit having an aperture fluidly connected to the air flow path that is positioned to redirect the flow path in a direction perpendicular to the air flow path in the elongated flexible conduit, and
(c) a valve affixed to the proximal end of the elongated flexible conduit and fluidly-connectable to a source of pressurized oxygen

2. The stylet of claim 1, wherein the elongated flexible conduit has an inner diameter and an outer diameter, wherein the outer diameter of the elongated flexible conduit of 3 to 8 mm.

3. The stylet of claim 2, wherein the inner diameter of the elongated flexible conduit is from 2 to 7 mm.

4. The stylet of claim 1, wherein the elongated flexible conduit comprises an elastomeric material that is not gas-permeable.

5. A system comprising the stylet of claim 1 telescopically received within an endotracheal tube.

6. The system of claim 5, wherein the stylet and endotracheal tube are contained in sterile packaging.
